(19) United States
(12) Patent Application Publication
(10) Pub. No.: US 2011/0124519 A1
(43) Pub. Date: May 26, 2011

(54) METHOD OF ASSESSING KIDNEY FUNCTION IN A HUMAN SUBJECT

(76) Inventors: Frank Walter Falkenberg, Witten (DE); Cormac Gerard Kilty, Sandycove (IE); Kerstin Schuster, Blackrock (IE)

(21) Appl. No.: 12/933,352

(22) PCT Filed: Mar. 18, 2008
(86) PCT No.: PCT/IE2008/000023

§ 371 Date: Dec. 22, 2010

(51) Int. Cl. C40B 30/04; G01N 33/573; G01N 33/53

(52) U.S. Cl. 506/9; 435/7.4; 435/7.9

(57) ABSTRACT

A method of assessing kidney function in a human subject to aid in the diagnosis of renal tubule diseases or conditions specific to a particular renal tubule region, comprises contacting a urine sample from said subject with a panel of capture molecules, each capture molecule being capable of binding to a specific biomarker from at least one of the four major regions of the renal tubule, in particular the proximal, distal, collecting duct and loop of Henle regions, the detection of one or more biomarkers in the urine sample being indicative of damage in a corresponding region of the renal tubule. The method facilitates inter alia the non-invasive detection of renal tubule damage and serves as a prognostic method to determine the level of tubule damage and monitor the effectiveness of therapeutic treatment.

### TECHNICAL FIELD

This invention relates to an assay format for the detection of urine biomarkers for use in determining the status of kidney function in a human subject.

### BACKGROUND ART

The classical methods of diagnosing kidney damage are based on the presence of symptoms such as reduced urine production, hypertension, fever and increased serum creatinine concentration. However, these symptoms occur when kidney damage or disease is established and they are inadequate for early detection.

Few diagnostic techniques are available for the identification of kidney damage. Current diagnostics methods to measure kidney function include monitoring urine for elevated protein levels such as albumin, or elevated serum creatinine, or calculating the glomerular filtration rate, which is a measure of the volume of fluid filtered from the kidney per unit time. More invasive and inconvenient tests include measuring the amount of nitrogen in the blood that comes from the waste product urea (Blood Urea Nitrogen test), or conducting renal biopsies. The above-mentioned diagnostic methods often are inadequate, since significant damage to the kidney can occur before symptoms are observed prior to diagnosis.

Early diagnosis is important in detecting renal damage which highlights a need to develop a method of diagnosing renal damage in the early stages, i.e., at a time in the course of the renal disease/damage where renal impairment is still minor and medical treatment may still be effective.

The main function of the kidney is as an excretory organ, eliminating excess fluid and waste metabolites from the blood. The kidneys regulate the body fluid level conserving or excreting water and electrolytes, as required. When damaged the kidneys lose their ability to regulate water and filter waste metabolites, which eventually leads to renal failure.

A human kidney contains approximately one million nephrons each consisting of coiled capillaries and tubules that lead to a collecting system.

It is known that in a normal kidney state no protein passes into the urine, because protein in the blood is too large to pass through the kidney filtration system. However, in a diseased or injured state the filtering ability of the kidneys can be damaged allowing protein to pass into the urine.

Injured kidney cells can “leak” specific biomolecules such as proteins, glycoproteins or metabolites thereof into the urine. Detecting and monitoring these specific biomolecular species in urine enables early diagnosis and accurate prognostic information about the stage of the disease/injury and the likely effect of any remedial therapies.

The identification of kidney biomarkers associated with defined locations in the kidney is required

In particular, there is a need for kidney biomarkers which are detectable and measurable in the urine, as urine is readily collected and results in minimal intervention and inconvenience for the patient.

The specificity of an assay is of key importance in its ability to distinguish between similar target molecules. It is known that biomarkers with absolute specificity are rare or perhaps nonexistent. Thus, there is a need to provide a combined assay system that can accurately detect biomarkers that are highly specific to individual regions of the nephron.

Detection of such specific biomarkers released into the urine in response to disease or injury could localise renal injury to exact sites.

Furthermore, a combination of different biomarkers for the specific regions of the nephron would allow for a greater understanding of the status of the whole kidney.

US 2006/0008804 A1 discloses methods for determining renal toxicity in individuals undergoing treatment with a cytotoxic agent and identifying candidate agents for use in the treatment of renal toxicity based on a comparison of gene expression of one or more genes selected from Calbindin-D28k, Kidney Injury Molecule-1 (KIM-1), Osteopontin (OPN), Epidermal Growth Factor (EGF), Clusterin/Testosterone-Repressed Prostate Message 2 (TRPM-2), Alpha-2u, Globulin Related-Protein (Alpha-2u), Complement Component 4 (C4), Vascular Endothelial Growth Factor (VEGF), Kidney-Specific Organic Anion Transporter-K 1 (OAT-K1), Aldolase A, Aldolase B and Podovin in a body sample obtained from said individuals relative to a control not subject to renal toxicity. Typically, the cytotoxic agent is an anti-cancer drug or immunosuppressant. It is stated that the body sample can be urine, but is particularly plasma. Although, the term individual is indicated to embrace humans, the experiments described in the specification were conducted on rats.

Biomarkers for kidney damage in a rat are not necessarily the same as those in a human.

For example, it is known that the isoenzyme πGST (pi glutathione S-transferase) is a marker for damage of the distal tubule in the human, whereas the isoenzyme μGST is a biomarker for damage of the distal tubule in the rat.

αGST is known to be a biomarker for damage in the proximal tubule in the human.

Falkenberg F. W. et al (1981) G. J. Hammerling and J. F. Kearney (Eds.), Elsevier North Holland, Amsterdam 148-155 disclose the development of immunological tests for kidney-derived urinary antigens for the determination of the corresponding antigens in serum and urine of patients.

Falkenberg F. W. et al (1981) Renal Physiol. 4:150-156 disclose the use of monoclonal antibodies to develop methods by which kidney-derived antigens can be detected in the urine of patients for diagnostic purposes.

Avrameas S. et al (1983) Elsevier Science Publishers Amsterdam, N.Y., Oxford 333-336 disclose the development of enzyme-immunoassays using monoclonal antibodies directed against antigen markers of defined renal structures. These monoclonal antibodies were prepared against human kidney antigens and characterized by immunofluorescence staining on human kidney slices. The antibodies displayed specificities for antigens in various parts of the nephron namely the glomerulus, proximal, and distal tubule, in blood vessels and in the interstitium.

Falkenberg F. W. et al (1983) Clinical Biochemistry 16:10-16 disclose the use of immunofluorescence staining to identify monoclonal antibodies which react with kidney derived soluble urinary antigens. A sandwich ELISA was developed using antibody PM II 9 C2 capable of detecting a urinary antigen in most urine samples.

Falkenberg F. W. et al (1984) Acta Medica, Rom/Italien 285-308 disclose the development of a number of sandwich ELISA tests using monoclonal antibodies specific to antigens localised in histologically defined regions of the nephron. These areas include the distal and proximal tubules. However, no assays have been developed for the collecting duct or loop of Henle regions of the nephron.

Pierard D. et al (1984) Proceedings of the 31^{st} Colloquium, New York, Pergamon 1047-1050 describe the use of monoclonal antibodies in a sandwich ELISA format to detect kidney derived components in urine. A total of fifteen antibodies were selected against various regions of the kidney which included distal and proximal tubules, glomeruli, all tubules and blood vessels. However, no antibodies were selected against collecting duct or loop of Henle regions.

Mai U. et al (1985) Transplantation Proceedings No. 6 2574-2575 disclose the preparation and characterization of monoclonal antibodies to antigens of the nephron of the human kidney and their use in sandwich ELISA tests for the determination of kidney-derived urinary antigens in urine. Thirteen such tests were developed, specific for antigens derived from cells of the proximal tubule, distal tubule, and for antigens localised over the entire length of the tubule system (pantubular antigens).

Falkenberg F. W. et al (1987) American Journal of Kidney Diseases IX: 129-137 disclose the use of monoclonal antibodies selected against the distal and proximal tubule to detect cellular fragments released in urine of patients treated with nephrotoxic drugs.

Holtmeier T. et al (1992) Transplant Monitoring 117-125 disclose the use of monoclonal antibodies to identify antigen from specific regions of the kidney via immunohistochemical staining. The antibodies used were specific for the proximal tubule and vascular endothelium. A third antibody (PM II 9 C2) was specific for Tamm-Horsfall protein (THP), a protein known to be excreted in healthy individuals and is abundant in mammalian urine.

At present, a panel of biomarkers for each of the four regions of the renal tubule is not available.

### DISCLOSURE OF THE INVENTION

Accordingly, the invention provides a method of assessing kidney function in a human subject, which method comprises contacting a urine sample from said subject with a panel of capture molecules, each capture molecule being capable of binding to a specific biomarker from at least one of the four major regions of the renal tubule, the detection of one or more biomarkers in the urine sample being indicative of damage in a corresponding region of the renal tubule.

By “capture molecule” herein is meant any molecule or portion thereof which binds reversibly or irreversibly to a said specific biomarker, so that said biomarker can be detected in the urine sample.

The method according to the invention provides a method for determining kidney function based on the detection of one or more biomarkers which are highly specific to regions of the renal tubule. Thus, the method according to the invention greatly facilitates the early diagnosis and treatment of kidney damage.

An advantage of the method according to the invention is that it enables screening for multiple biomarkers. These biomarkers are located in specific regions of the renal tubule and their detection correlates to damage in that particular region.

By “panel of capture molecules” herein is meant a panel of two or more capture molecules capable of binding to two or more specific biomarkers from at least one of the four major regions of the renal tubule.

Thus, the method according to the invention will aid in the diagnosis of renal tubule diseases or conditions specific to a particular region thereof and will help to monitor kidney status and treatment.

The method according to the invention also enables detection of kidney biomarkers in a single urine sample.

The method according to the invention also allows for the detection of multiple biomarkers from the same urine sample. Thus, instead of processing separate samples to test for two or more markers, the same sample can be processed using a panel of single assays.

The method according to the invention thus allows for patient samples to be processed with higher speed and efficiency and requiring less patient sample than previously required.

The method according to the invention also facilitates the non-invasive detection of renal tubule damage and serves as a prognostic method to determine the level of kidney damage and monitor biomarker presence in urine.

A further advantage of the present invention is that it permits one to diagnose, or aid in the diagnosis of, kidney damage or to otherwise make a negative diagnosis.

The method according to the invention also improves on the capability to diagnose, detect and monitor kidney damage using a reliable and non-invasive technique.

Preferably, the method is capable of detecting less than 60% damage in a region of the renal tubule.

Thus, the method according to the invention is able to detect kidney damage before current diagnostic techniques such as those based on serum creatinine, which is normally detected when there is up to 60% damage in a region of the renal tubule.

Further, preferably, the method is capable of detecting damage of the order of 5-10% in a region of the renal tubule.

The present invention enables kidney damage over the entire renal tubule to be determined at an earlier stage than current methods, thereby permitting early diagnosis and medical intervention.

By kidney function herein is included kidney damage following physical, microbial, for example due to infection or sepsis, or toxicological insult.

According to one embodiment of the invention, the or each biomarker is detected by immunoassay.

Preferably, the capture molecule is an antibody

Further, preferably, the antibody is a monoclonal antibody.

The detection of the biomarker in accordance with the invention can be solely by immunoassay.

A particular requirement of the method according to the invention is antibodies with the requisite affinity and specificity for their biomarker targets.

In one embodiment of the invention, the biomarkers include alpha glutathione S-transferase (αGST) which is specific to the proximal tubule, pi glutathione S-transferase (πGST) which is specific to the distal tubule, and markers specific to the kidney sites loop of Henle and the collecting duct.

According to one embodiment of the invention, the antibody is monospecific for alpha glutathione S-transferase (αGST) specific to the proximal region of the renal tubule.

A hybridoma producing a monoclonal antibody specific for αGST, 5B11, which is specific for the proximal region of the renal tubule, was deposited with Deutsche Sammlung von Mikroorganismen und Zellkulturen GmbH (DSMZ) on Feb. 27, 2008, and accorded the number DSM ACC2886.

According to a further embodiment of the invention, the antibody is monospecific for pi glutathione S-transferase (πGST) specific to the distal region of the renal tubule.

A hybridoma producing a monoclonal antibody specific for πGST, N5D12, which is specific for the distal region of the renal tubule was deposited with DSMZ on Feb. 27, 2008 and accorded the number DSM ACC2884.

According to a further embodiment of the invention the antibody is monospecific for antigen specific to the collecting duct region of the renal tubule.

A hybridoma producing a monoclonal antibody specific for the collecting duct region of the renal tubule, HuPap VII 2B11, was deposited with DSMZ on Feb. 27, 2008 and accorded the number DSM ACC2883.

According to a still further embodiment of the invention the antibody is monospecific for antigen specific to the loop of Henle region of the renal tubule.

A hybridoma producing a monoclonal antibody specific for the loop of Henle region of the renal tubule, PapX5C10, was deposited with DSMZ on Feb. 27, 2008 and accorded the number DSM ACC2885.

Surprisingly, the monoclonal antibody specific for the loop of Henle region in the human cross-reacts with an antibody which is specific for the collecting duct in the rat.

It will be appreciated that polyclonal antibodies that demonstrate the requisite specificity can also be used in the method according to the invention.

According to one embodiment of the invention, the detection of the antibody-biomarker complex comprises contacting the antibody (the primary antibody)-biomarker complex with a second antibody (the secondary antibody).

Immunoassay techniques for use in accordance with the invention include sandwich, competitive, non-competitive, direct and indirect assays.

A detection enzyme may be linked directly to the primary antibody or introduced through the secondary antibody that recognises the primary antibody.

Preferably, the second antibody is a labelled antibody and the detection of the presence of biomarker-antibody complex is effected by detecting the label on the antibody.

The label for the antibody may also be an entity detectable by biochemical, photochemical, immunological, spectroscopic, biophysical or any chemical means.

Preferably, the second antibody label is selected from the group consisting of an affinity label, biotin, a chromophore, a colloidal metal, dioxigenin, a dye, an enzyme, an enzyme substrate, a fluorophore, a lumiphore, a magnetic particle, a metabolite, a radioisotope and streptavidin.

The or each biomarker can be detected using an enzyme immunoassay, more especially a sandwich enzyme immunoassay.

The method according to the invention allows for any biomarker present in the sample to form a complex with its corresponding antibody. Unbound proteins are removed by washing, and a labelled second antibody is allowed to bind to its corresponding biomarker forming an antibody-biomarker complex, signalling the presence of a biomarker in the sample.

In one particular embodiment of the invention the determination of the antibody-biomarker complex is carried out by a competition immunoassay.

According to a further embodiment of the invention, a said biomarker is detected enzymatically.

It will be appreciated that the capture molecule for αGST, being an enzyme, can be a substrate or co-factor for αGST.

It will also be appreciated that the capture molecule for πGST, being an enzyme, can also be a substrate or co-factor for πGST.

Assays carried out in accordance with the invention can be multiplexed to simultaneously measure multiple biomarkers in a single sample in a manner known per se.

Muliplex solid support platforms include, for example, microtitre wells, biochips, nitrocellulose membranes, nylon membranes, Polyvinylidene Fluoride (PVDF) membranes or glass or plastic plates.

According to one embodiment of the invention the capture molecules are bound to a microtitre plate.

An advantage of the method according to the invention is that having the capture molecules of the assay format immobilised on a solid surface enables the separation of bound from unbound species during the assay. This ability to wash away non-specifically bound materials makes the assay a powerful tool for measuring specific biomarkers within a crude sample.

According to an alternative embodiment, the capture molecules are bound to a biochip array.

An advantage of the use of biochip arrays is that it allows for the high throughput analysis of a large number of biomarkers.

The method according to the invention can be used in the diagnosis of diseases known to be associated with specific regions of the renal tubule.

The method according to the invention improves on the capability to diagnose diseases or functional disorders known to be associated with specific regions of the renal tubule.

The method according to the invention can also be used for assessing the effectiveness of therapeutic treatments.

An advantage of the present invention is that the level of tubule damage can be assessed routinely to determine if therapeutic treatment is effective.

The method according to the invention can also be used for assessing the tolerance of a subject to a specific pharmaceutical treatment.

An advantage of the method according to the invention is that it can be used to maximise the effectiveness of different therapeutic treatments, whilst simultaneously reducing the possibility of toxicity side effects.

It will be appreciated that the method according to the invention can be used for assessing the safety of clinical trials.

It will be appreciated that individuals have different urinary biomarker reference baseline levels. Therefore, post-operative or post-treatment results should be considered in relation to the patient's pre-operative or pre-treatment reference baseline biomarker level, as appropriate.

According to one embodiment of the invention, the invention provides a test kit or assay comprising a panel of capture molecules, each capture molecule being capable of binding to a specific biomarker from at least one of the four major regions of the renal tubule.

### BRIEF DESCRIPTION OF THE DRAWINGS

FIG. 1 is an immunohistochemistry stain photograph of Human Renal Tissue stained with 5B11 (Antibody against Proximal Tubule);

FIG. 2 is an immunohistochemistry stain photograph of Human Renal Tissue stained with N5D12 (Antibody against Distal Tubule);

FIG. 3 is an immunohistochemistry stain photograph of Human Renal Tissue stained with HuPap VII 2B11 (Antibody against collecting duct);

FIG. 4 is an immunohistochemistry stain photograph of Human Renal Tissue stained with PapX5C10 (Antibody against loop of Henle);

FIG. 5 is an image of an immunoblot using gold-conjugated antibodies with PapX5C10 (Antibody against loop of Henle); and

FIG. 6 is an image of an immunoblot using gold-conjugated antibodies with HuPap VII 2B11 (antibody against collecting duct).

### MODES FOR CARRYING OUT THE INVENTION

The invention will be further illustrated by the following Examples

### Preparatory Example A

### Purification of Human αGST and πGST

πGST was purified from human placenta and αGST was purified from human liver by affinity chromatography. Placenta and liver are good sources of πGST and αGST, respectively. The presence of αGST and πGST in the urine can only come from leakage from the kidneys due to kidney damage. Precise details of the purification procedure are as follows:
(a) Human tissue was homogenised for 2 min in homogenisation buffer, at a ratio of one part tissue to four parts buffer, using a Waring (Waring is a Trade Mark) blender. The homogenisation buffer at pH 7.2 had the following composition:
   250 mM Sucrose/10 mM Sodium Phosphate/2 mM EDTA 20 mM Tris-HCl.

1 μg/ml Leupeptin, 1 μg/ml Pepstatin, 0.2 μg/ml phenylmethanesulphonyl fluoride (PMSF) and 2 μg/ml Aprotinin.
(b) The tissue homogenate was centrifuged in two steps, first at 2000 g for 15 min. The supernatant was decanted and centrifuged for a second time at 20,000 g for 60 min at 5° C.
(c) The supernatant was then loaded on a Glutathione (GSH)-Sepharose Affinity column previously equilibrated in 20 mM Tris-HCl with 200 mM NaCl, pH 7.8. Equilibration buffer was reapplied to elute unbound protein. Finally 50 mM Tris-HCl pH 8 containing 10 mM GSH was used to elute bound GST from the affinity column.
(d) The eluted material was then dialysed against phosphate buffered saline (PBS) pH 7.2-7.4.
(e) The αGST and πGST were stored in aliquots at −20° C. until required for use.

### Preparatory Example B

### Production and Purification of Human Recombinant GST Isoenzymes Vector Information

The plasmid pChromo-hGSTpi is a 3272 bp vector derived from pUC19 developed to facilitate the expression of recombinant human πGST. The basis of the recombinant αGST expression-system is a 3425 bp vector derived from pRSETa. These E. coli expressed recombinant GST proteins lack any additional “vector encoded” amino acids.

The presence of the GSTs in the constructs has been confirmed by DNA sequencing. A clustalW alignment of forward and reverse DNA sequence compares the sequence with published cDNA sequences for human GST and confirms that the recombinant protein and native protein are identical in amino acid sequence.

### Expression and Purification of Recombinant GST Isoenzymes

The transformed E. coli BL-21 cells were grown by picking a single colony and inoculating starter cultures of 5 ml of Luria-Bertani (LB) broth. The cultures were grown overnight at 37° C. with vigorous shaking at around 300 rpm.

The starter cultures were diluted 1/100 into selective LB medium containing 100 μg/ml ampicillin (next morning) and again grown overnight at 37° C. with vigorous shaking at ˜300 rpm until the culture reached an optical density (O.D.) at 600 nm of 1.2.

The cells were induced with 1 mM isopropylthiogalactose (IPTG) and incubated for 24 h at 37° C. with vigorous shaking (˜300 rpm).

The bacterial cells were harvested by centrifugation at 6,000×g for 15 min at 4° C. The resulting bacterial pellet was frozen and re-thawed before suspension in PBS pH 7.5 with 10 μg/l lysozyme and incubated for 60 min on ice. Total cell lysates were obtained by sonication for 5 min at 50% duty cycle with power output setting at 5 using a Branson Sonifier 250.

Sonicated cells were centrifuged at 19,000×g for 45 min at 4° C. and the supernatant containing recombinant GST removed promptly. The recombinant GST protein was purified by affinity chromatography using 6% CL-Gluthathione ChroMatrix™ resin according to manufacturer's instructions.

GSH from elution buffer was removed by dialysis or gel filtration.

The purity of the protein was checked via SDS electrophoresis.

### Preparatory Example C

### Polyclonal Antibody Production and Purification

Purified human GST (π or α, native or recombinant) was injected into New Zealand White rabbits subcutaneously (s.c.) according to the time schedule given below and serum evaluated for anti-GST reactivity. Once the IgG [anti-human GST] titre was sufficient as determined by semi-quantitative dot blot analysis, the animals were exsanguinated and serum collected. Total IgG was purified from rabbit serum by Protein A affinity chromatography and was used for conjugation to horseradish peroxidase (HRP) or plate coating.

### Immunisation Schedule (General)

Day 1: A test bleed of 12-15 ml of preserum was taken from the ear of the rabbit. 0.5 ml of human GST antigen (25 μg) was mixed with an equal volume of Freund's Complete Adjuvant. The mixture of antigen and adjuvant was homogenised to ensure a good emulsion. This mixture was then injected intramuscularly into the hind legs.

Day 28: A boost injection was given to the rabbit. 0.5 ml antigen (25 μg) was mixed with an equal volume of Freund's Incomplete Adjuvant. The antigen/adjuvant mixture was homogenised to ensure a good emulsion. This mixture was then injected 2×0.25 ml intramuscularly and 2×0.25 ml subcutaneously into the flank over the ribs, which had been slightly shaved before the injection.

Day 35: A test bleed of 4 ml of blood was taken from the ear of the rabbit.

Day 56: A second boost was given to the rabbit as described on Day 28.

Day 63: A test bleed of 4 ml of blood was taken from the rabbit's ear.

Day 84: A third boost was given to the rabbit as described on Day 28.

Day 91: A test bleed of 4 ml of blood was taken from the rabbit's ear.

Day 112: A fourth boost was given to the rabbit as described on Day 28.

Day 119: A production bleed of 15-25 ml was taken.

Day 120: The rabbit was sacrificed and as much blood as possible collected.

### Preparatory Example D

### Monoclonal Antibody Production and Purification

Monoclonal IgG [anti-human GST] clones were obtained from The University Hospital Nijmegen, The Netherlands and the University Wisconsin, US.

Monoclonal collecting duct and loop of Henle antibodies were produced as follows:

(a) Production of Monoclonal Antibodies in the miniPERM Bioreactor (miniPERM is a Trade Mark)

Monoclonal antibodies are produced under sterile conditions in the miniPERM™ bioreactor.

Function and handling of the miniPERM™ bioreactor is described in Falkenberg, F. W. et al. ((1995) J Immunol Methods 179:13-29).

miniPERM™ (mP) harvests were collected and centrifuged under sterile conditions.

The sterile mP supernatants harvested were stored frozen.

(b) Preparation of the mP Supernatant Sample for DEAE Ion Exchange Chromatography

For the purification procedure 1-3 mP harvests (about 25-30 ml each) were thawed and pooled.

If required, sodium azide can be added to a final concentration of 0.01%.

The pooled mP supernatants were applied to a 430 ml Superdex (Superdex is a Trade Mark) 30 column (26 mm diameter, 880 mm length).

The Superdex™ 30 column was run with the 0.02 M triethanolamine (TEA) buffer, pH 7.9, required as the starting buffer for the diethylamino ethanol (DEAE) column fractionation.

The fractions of the first peak contained the eluted antibodies and were pooled.

(c) DEAE Ion Exchange Column Chromatography

For DEAE ion exchange chromatography a column (20 mm diameter, 150 mm long, total volume 47 ml) filled with TSK™ gel DEAE-5PW anion exchange material (13 μm particle size) was used.

Before use the column was washed with 3 column volumes of the DEAE chromatography starting buffer (0.02M TEA, pH 7.9).

Then the pooled Superdex 30™ fractions were applied to the DEAE column in a Fast Protein Liquid Chromatography (FPLC) system.

After washing with 3 column volumes of starting buffer (to remove unbound cationic proteins) the column was eluted by application of a NaCl gradient ranging from 0.000 to 1.000M NaCl.

The gradient was not kept straight over the whole NaCl concentration range.

The steepness of the gradient can be adapted to the ionic properties (IP) of the monoclonal antibodies concerned.

In general the following gradient profile was used:
from 0.0 to 50 mM (2 column volumes) the gradient is kept steep.
from 50 to 200 mM (9 column volumes) the gradient is kept flat.
from 200 to 300 mM (2 column volumes) the gradient is kept steep.
from 300 to 1.000 mM (0.01 volumes) the gradient is kept very steep.

The gradient profile can be adapted as required.

If required, the elution buffers can be supplemented with sodium azide (0.01%).

(d) Treatment of the Eluted Monoclonal Antibodies

Monoclonal antibodies were normally eluted in the 80-120 mM NaCl gradient range.

The antibody-containing peak fractions were identified by specific tests (indirect immunofluorescence, ELISA) and pooled.

If required the pooled fractions were concentrated by ultrafiltration on a 10 kDa filter.

Finally, the monoclonal antibodies were sterile filtered, and OD280 was determined.

In case the eluted monoclonal antibodies were still contaminated by fetal calf serum (FCS) proteins, a re-chromatography was performed under the same conditions as indicated above.

In order to remove contaminating proteins from the columns, the columns were regularly treated by several cycles of washing with 0.5 M NaOH.

For storage the columns were kept in buffer with 0.01% sodium azide.

### Hybridoma Generation

Hybridoma generation was performed following—in principle—the procedure described by Köhler, G and Milstein, C ((1974) Nature 256:495).

NS0 non-secretor HGPRT deficient myeloma cells were obtained from Dr. Milstein. Polyethylene-glycol (PEG) was used as a fusiogen following the procedure described by Goding, J. W. (1983) Monoclonal Antibodies: Principles and Practice. Academic Press London, New York.

In Detail:

NSO Myeloma cells were cultured in Dulbecco's Modified Eagle's Medium (DMEM) containing Horse Serum (HS) and harvested before they reached confluency.

The cells were washed and resuspended in serum-free medium.

Immunized mice were sacrificed and their spleens were excised.

Spleen cell suspensions were prepared, washed and resuspended in serum-free medium.

Then the spleen cell suspension and the myeloma cell suspension were mixed in a ratio of 5:1 (spleen cells: myeloma cells).

The mixture was centrifuged, and the supernatant removed as completely as possible.

After addition of the fusiogen (PEG) and careful shaking for 1 min, the PEG was diluted by addition of serum-free medium.

The cell suspension was then centrifuged (200×G) and the PEG-containing supernatant was discarded.

The cells were washed several times with serum-containing medium.

The cell suspension was then adjusted to 100,000 cells per ml and aliquots of 100 μl, containing 10,000 cells, were seeded into the wells of 96 well tissue culture plates.

After incubation for 24 h in a tissue culture incubator (37° C., 8% CO₂) 100 μl of hypoxanthine/aminopterin/thymidine (HAT) selection medium (GIBCO) was added to each well.

After 5 days of culture, the spent medium was carefully removed from the wells and replaced by 200 μl of fresh HAT selection medium.

As soon as the culture medium in the wells became yellow (indicating rapid cell proliferation) the supernatants were recovered and used for testing for the presence of the desired antibodies.

The supernatants containing monoclonal antibodies were tested on fixed or frozen human kidney slices prepared with an appropriate microtome.

For detection of monoclonal antibodies reacting with antigens in the renal slices indirect immunofluorescence with fluorochrome-labeled anti mouse Ig antibodies (absorbed with serum from various species) was performed.

Hybrid mixtures containing antibodies with the desired specificities were then cloned, either by limiting dilution or by soft agar cloning.

Before application in the cloning procedure the cells were slowly adapted to growth in medium without HAT.

Cell clones observed in the cloning plates were tested using indirect immunofluorescence on frozen kidney slices.

Clonal cell cultures producing antibodies with the desired specificities were expanded, and the cells were stored frozen in liquid nitrogen.

For antibody production, the cells were adapted to culture in DMEM containing FCS instead of HS.

### Preparatory Example E

### Preparation of Renal Tissue Sections on a Cryomicrotome

Human renal tissue specimens were stored at −80° C. covered with TissueTec™. Prior to analysis, the temperature of the tissue specimens was raised to −20° C. over a period of 30 min in the cryomicrotome, in order to reach the optimal temperature for slice preparation.

The tissue specimens were fixed in the object holder and adjusted so that transverse sections were prepared. The thickness of the slices was 5-7 μm. Slices were drawn on object slides. In order to achieve proper fixation to the glass slides the sections were stored overnight at 4° C. and could be used for one week.

### Indirect Immunofluorescence Staining

Reagents required
Tissue slices (5-7 μm)
Unlabelled primary antibodies (purified, miniPERM™ supernatants or cell culture supernatant)
Fluorochrome-labelled secondary antibodies

### Procedure

(a) Cell culture supernatants were used undiluted. Purified antibodies were diluted in PBS to a final concentration of 1-10 μg/ml. miniPERM™ supernatants were applied in dilutions from 1:30 to 1:1000. 50-100 μl of the solution of the unlabelled antibodies was required depending on the size of the sections.

(b) The tissue slices were incubated with the unlabelled antibody solution for 60 min. in a humid box protected from light.

(c) After incubation the slices were washed three times in PBS.

(d) The dilution of the fluorescence-labelled antibodies had to be predetermined. In general, the Alexa Fluor-labelled secondary antibodies (Invitrogen) were diluted 1:150 or 1:300 in PBS. 50-100 μl of the antibody solution was required.

(e) The tissue slices were covered with the antibody solution and incubated for 60 min. in a humid box protected from light.

(f) The tissue slices were exposed to three washing cycles in PBS.

(g) The slides were carefully dried around the tissue which was then covered with 20 μl of 50% PBS/Glycerol. A cover glass was applied to the slide.

(h) The slides were carefully wiped dry. Care had to be taken not to damage the tissue slice. Then 20 μl of 50% glycerol in PBS was pipetted onto the tissue slice. A cover glass was applied to the slide.

### Preparation of and Fractionating Kidney Extract

### Preparation Human Kidney Extract

Human kidney extracts were prepared as follows:

(a) 5 grams of 10 μm slices of the frozen human kidney pieces were prepared as described above. The slices were suspended in 10 ml of PBS.

(b) The tissue was then homogenized with Ultra-Turrax (Janke & Kunke, Germany). The homogenate was subjected to two cycles of freezing in liquid nitrogen, thawing in 37° C. H₂0 and 1 min. ultrasonic treatment. The homogenates were then centrifuged in Eppendorf tubes at 15,000 rpm.

(c) Turbidity in the supernatant was removed by filtering through a 5.0 μm filter followed by filtration through a 0.2 μm sterile filter.

(d) The extract was aliquoted and kept frozen at −20° C. The pellets of the extract were resuspended and stored frozen (−20° C.)

### Preparatory Example F

### Immunoblotting

All polyclonal and monoclonal GST IgGs for use in the following Examples were checked for πGST and αGST reactivity and potential cross-reactivity, via the following immunoblot combinations:

(a) Rabbit IgG [anti-human πGST and αGST] was used to probe nitrocellulose membranes containing immobilised human α and πGST.

(b) Murine IgG [anti-human πGST and αGST] was used to probe nitrocellulose membranes containing immobilised human α and πGST.

The method used for immunoblot detection was as follows:

(i) Human α and πGST (0.5 μg/track) were electrophoresed on 15% SDS-PAGE with molecular weight markers also included.

(ii) After electrophoresis, the polyacrylamide gel was cut and one half stained for protein while the remainder was used for electrophoretic transfer onto nitrocellulose.

(iii) After electrophoretic transfer, the nitrocellulose membranes were blocked for 1 h with 5% (w/v) Marvel (Marvel is a Trade Mark) in PBS 0.05% (w/v) Tween-20 (PBST)-blocking buffer.

(iv) The following solutions were then prepared:

Rabbit IgG [anti-human πGST] in 1% (w/v) Marvel in PBST

Rabbit IgG [anti-human αGST] in 1% (w/v) Marvel in PBST

Murine IgG [anti-human πGST] in 1% (w/v) Marvel in PBST

Murine IgG [anti-human αGST] in 1% (w/v) Marvel in PBST

(v) The antibody solutions were individually added to nitrocellulose membranes containing immobilized human αGST and πGST, once blocking buffer was decanted. Incubation with antibody solution was allowed to proceed for 1 h.

(vi) The nitrocellulose membranes were then washed in PBST (2× for 5 min each).

(vi) Anti-rabbit IgG-horseradish peroxidase (HRP) conjugate was then prepared ( 1/1000 in 1% (w/v) Marvel in PBST and added to rabbit IgG antibody complexes. Anti-murine IgG-HRP conjugate was also prepared ( 1/1000) and added to murine IgG antibody complexes.

After 1 h incubation with anti-species conjugates, the reagents were discarded and the membranes washed in PBST with % (w/v) Marvel.

(vii) Diaminobenzidine substrate was then prepared and added to the membrane. A positive reaction was indicated by a brown precipitate on the nitrocellulose membrane.

### Preparatory Example G

### Anti-GST IgG-HRP Conjugate Synthesis

Anti-GST IgG-HRP conjugates were synthesised using thioether conjugation methodology. (Duncan, R. J. S., et al., (1983); Anal. Biochem. 132, 68-73). Reactive maleimide groups were introduced onto IgG molecules using SMCC (succinimidyl 4-(N-maleimidomethyl)cyclohexane 1-carboxylate) and masked sulphydryl groups were linked to HRP. After a demasking step to produce reactive sulphydryl groups, the maleimide-activated IgG and HRP-SH were mixed together and allowed to react for 4.5 h. The resultant IgG-HRP conjugate formed by covalent thioether linkage, was brought to 50% (v/v) glycerol and stored at −20° C. for use in the EIA.

### Preparatory Example H

### Procedure for the Detection of Collecting Duct and Loop of Henle Protein in Urine

Protocol for Dot Immunoblotting Using Gold-Conjugate Antibodies

(a) Human renal extract was diluted 1/10, 1/50 and 1/100 in PBS buffer. 1 μl volumes of extract dilutions and 1 μl of test urine samples were spotted onto nitrocellulose membranes and allowed to air-dry. Membranes were stored overnight at 2-8° C. Membranes were blocked by incubation in PBS containing 4% (w/v) BSA for 30 min at 37° C.

(b) Antibodies PAPX5C10 (IgG1) and HuPAPVII2B11 (IgM) were diluted 1/200 in PBS containing 0.8% (w/v) BSA. Antibody solutions were incubated with separate nitrocellulose blots for 2 hours at room temperature on shaking platform.

(c) Membranes were washed three times with PBS containing 4% (w/v) BSA (10 min-per wash with shaking).

(d) Gold-labelled secondary conjugate antibodies were diluted 1/100 in PBS containing 0.8% (w/v) BSA, 0.1% (w/v) gelatin hydrolysate and 0.05% (v/v) Tween® 20 in PBS and incubated with nitrocellulose membranes for 2 hours at room temperature on a shaking platform as follows:
(i) Goat-anti mouse-IgG-gold conjugate antibody was added to membranes previously incubated with PAPXC10 IgG
(ii) Goat-anti mouse-IgM-gold conjugate antibody was added to membrane previously incubated with HuPAPVII2B 11 IgM.

(e) After 2 hours incubation, the membranes were washed as follows:
(i) 3×5 min washes with 0.8% (w/v) BSA in PBS on shaking platform.
(ii) 2×5 min washes with PBS on shaking platform.
(iii) 1×10 min incubation in 1% (v/v) glutaraldehyde in PBS (no shaking).
(iv) 2×5 min washes with dH₂O on shaking platform.
(v) 1×5 min wash with 50 mM EDTA pH 4.5 on shaking platform.

(f) Nitrocellulose membranes were incubated for 30 min. at room temperature with BB International Silver Enhancing Kit (Cat. Code SEKL15) following the manufacturer's instructions. Development was performed without shaking.

(g) Nitrocellulose membranes were extensively washed with distilled water.

### Preparatory Example I

### Sandwich Enzyme Immunoassay

### αGST

The format of the immunoassay for the quantitative detection of human αGST was a conventional sandwich format as described in our EP 0 880 700 B and described further below. A kit for performing the assay is available from Biotrin International Limited under the Trade Mark NEPHKIT. NEPHKIT® Alpha GST EIA Cat No. BIO66NEPHA.

(a) A Nunc Maxisorp (Nunc Maxisorp is a Trade Mark) microtitre plate was direct coated with rabbit polyclonal IgG [anti-human αGST] (referred to in Preparatory Example C).

(b) Human αGST, purified from liver as described in Preparatory Example A or recombinant protein as described in Example B, was used as the assay calibrator.

(c) Polyclonal IgG [anti-human αGST]-HRP conjugates, in association with tetramethylbenzidine substrate (TMB), were used to facilitate detection of captured/immobilised αGST.

(d) The enzyme reaction was stopped by the addition of 1N H₂ SO₄ and the absorbance measured at 450 nm using 630 nm as a reference wavelength. Colour intensity was proportional to αGST concentration and after generating a plot of A₄₅₀/630 nm versus concentration (μg/L), the concentration of unknown samples can be determined using a standard curve. Total assay time was less than 2.5 h.

The total assay time was found to be 2 h 15 min and assay conditions included microtitre plate shaking at fixed temperature during the sample and conjugate incubation steps, respectively. The TMB substrate incubation required fixed temperature conditions only.

### Preparatory Example J

### πGST

The format of the immunoassay for the quantitative detection of human πGST was a conventional sandwich format available in kit form from Biotrin International Limited—Cat. No. BI085 as described further below.

(a) A Nunc Maxisorp (Nunc Maxisorp is a Trade Mark) microtitre plate was coated with murine monoclonal IgG [anti-human πGST] (referred to in Preparatory Example F) immobilised via goat F(ab)₂ fragments [anti-mouse IgG]. This method of antibody coating serves to orientate Mab binding sites and also improves assay sensitivity by minimising adherence-induced denaturation of the capture antibody.

(b) Human πGST, purified from placenta as described in Preparatory Example A or recombinant protein as described in Example B, was used as the assay calibrator.

(c) IgG [anti-human πGST]-HRP conjugates, in association with TMB substrate, were used to facilitate detection of captured/immobilised πGST.

(d) The enzyme reaction was stopped by the addition of 1N H₂ SO₄ and the absorbance measured at 450 nm using 630 nm as a reference wavelength. Colour intensity was proportional to πGST concentration and after generating a plot of A₄₅₀/630 nm versus concentration (μg/L) for standard samples, the concentration of unknown samples can be determined using a standard curve. Total assay time was less than 2.5 h.

The total assay time was found to be 2 h 15 min and assay conditions included microtitre plate shaking at fixed temperature during the sample and conjugate incubation steps, respectively. The TMB substrate incubation required fixed temperature conditions only.

### Example 1

### Immunofluorescence Stains of Kidney Tubule Slices Demonstrating Antibody Specificity

Immunofluorescence stains of each of the four main regions of the human renal tubule using fluorochrome-labelled secondary antibodies and immunofluorescence staining technique were performed

Human tissue material was obtained from kidneys of patients suffering from renal carcinoma. The kidneys were removed by nephrectomy.

Parts of the kidney free of tumour were excised, covered with TissueTec™, shock frozen in liquid nitrogen and stored frozen at −85° C. 5-7 μm thin kidney slices were prepared in a cryomicrotome (Microm, Germany) and mounted on microscopic slides.

The primary antibodies used were antibody 5B11, binding to antigen in the proximal tubule (DSM ACC2886); antibody N5D12, binding to antigen in the distal tubule (DSM ACC2884); antibody HuPAPVII2B11, binding to antigen in the collecting duct (DSM ACC2883) and antibody PapX5C10, binding to antigen in the loop of Henle (DSM ACC2885).

50 μl samples of the monoclonal antibodies tested were carefully distributed over the kidney slices in pre-determined dilutions. After 60 minutes of incubation in an incubation chamber at room temperature, the slides with the slices were washed in PBS.

50 μl of a secondary fluorochrome-labelled goat anti-mouse IgG (heavy & light chain-specific), goat anti-mouse IgG (IgG subclass heavy chain-specific) or goat anti-mouse IgM (μ.-chain-specific) antibody were applied to the slice.

The slide was incubated for 60 minutes at room temperature in an incubation chamber protected from light. The slide was washed with PBS to remove excess fluorochrome labelled antibodies.

Approximately 50 μl of 10% glycerol in PBS were added to the slice, and a cover slip was laid over the slice and fixed with glue.

The secondary antibodies used had been extensively absorbed with human and other species serum proteins. Two fluorochromes were used, AlexaFluor 488 (green fluorescence) and AlexaFluor 495 (red fluorescence).

After staining, the tissue slices were inspected under a Nikon microscope equipped with a fluorescence illuminator. Pictures were taken with a 12 megapixel digital camera.

In order to get an overview of the reaction of the antibodies in a broader section of the kidney, series of pictures were taken covering a stretch of tissue extending from the cortex to the medulla. The individual pictures were assembled applying PhotoShop™ software.

### Results

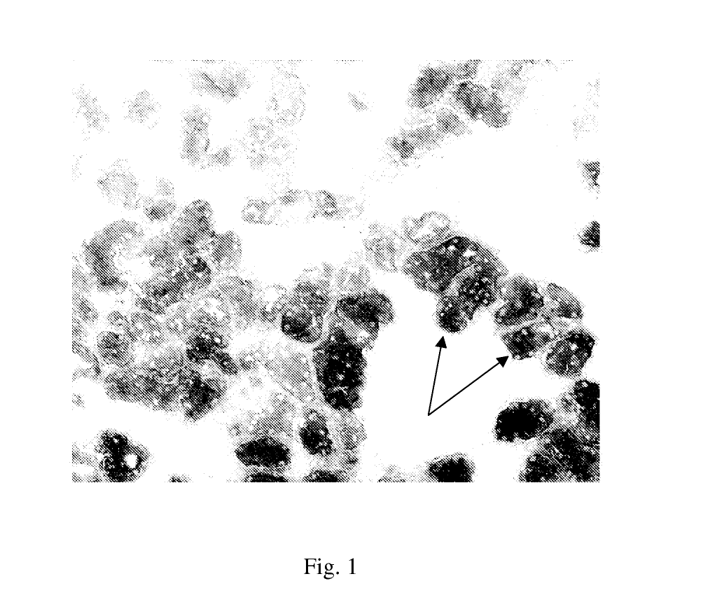

FIG. 1 shows the localisation of antibody 5B11 binding to antigen in the proximal tubule epithelial cells as depicted by the arrows. The specific fluorescence of proximal tubules is shown. Typically for proximal tubules, the tubules are grouped around glomerules.

Distal tubules and glomeruli show no staining. The specificity exhibited by the antibody 5B11 allows effective absolute discrimination between the proximal region and other regions of the renal tubule.

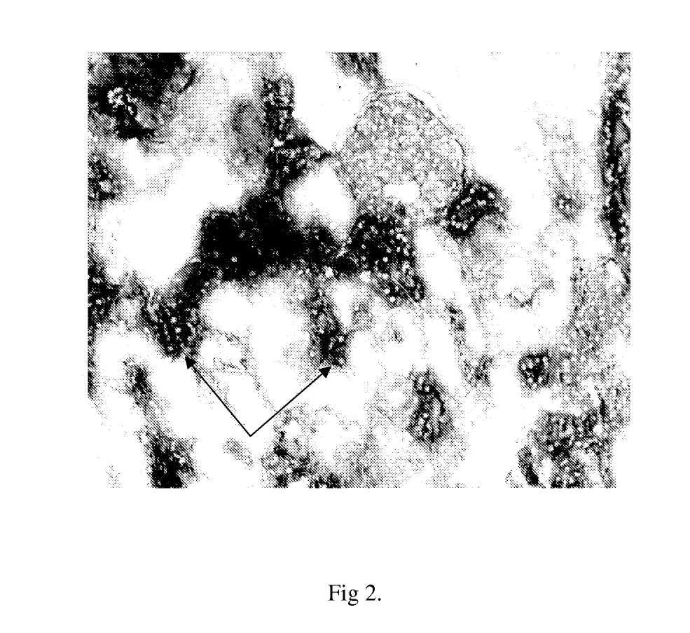

FIG. 2 shows the localisation of antibody N5D12 binding to antigen in the distal tubule epithelial cells as depicted by the arrows. The specific staining of distal tubules in the cortex of human kidney is shown. The proximal tubules and glomeruli show no staining.

The specificity exhibited by the antibody N5D12 allows effective absolute discrimination between the distal region and other regions of the renal tubule.

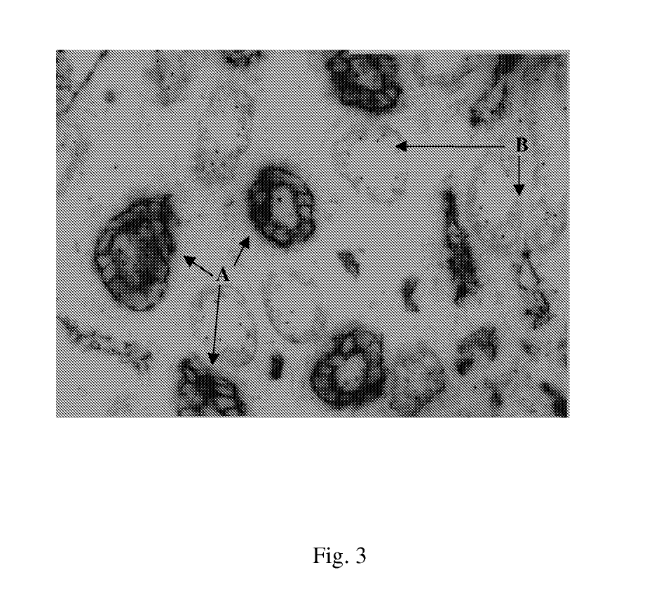

FIG. 3 shows the localisation of antibody HuPAPVII2B 11 binding to antigen in the collecting duct epithelial cells. Arrows A show the specific reaction of HuPapVII2B11 with collecting ducts in the medulla of human kidney and arrows B show loop of Henle epithelial cells. Proximal and distal tubules show no staining. The specificity exhibited by the antibody HuPAPVII2B 11 allows effective absolute discrimination between the collecting duct region and other regions of the renal tubule.

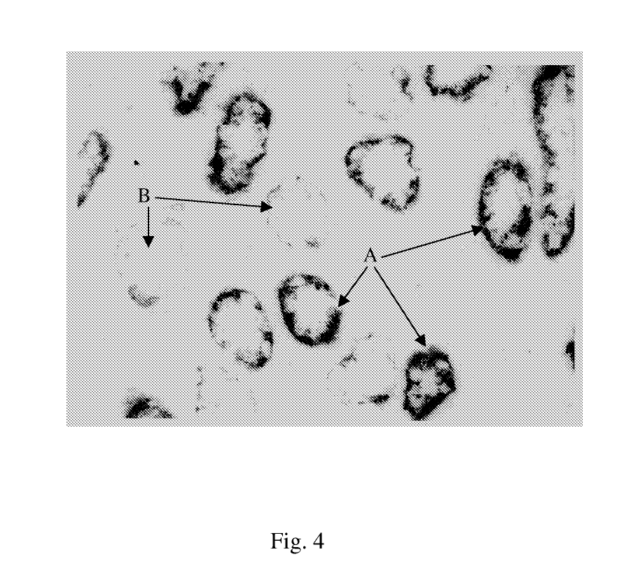

FIG. 4 shows the localisation of antibody PapX5C10 binding to antigen in the loop of Henle epithelial cells. Arrows A show specific staining of loops of Henle in the medulla of human kidney and arrows B show collecting duct epithelial cells. The specificity exhibited by the antibody PapX5C10 allows effective absolute discrimination between the loop of Henle region and other regions of the renal tubule.

### Example 2

### Screening of Urine Samples from ICU Patients for Evidence of Renal Tubule Damage

Human kidney extracts were diluted ⅕ and 1/50 in 1% w/v bovine serum albumin (BSA)/PBS buffer as described in Preparatory Example H. These extracts were used as a control positive to confirm the antibodies PapX5C10 and HuPAPVII2B11 bound to kidney tubule antigen and as immunoblot reaction standards.

The α and π-GST immunoassays were performed using the Alpha GST EIA Kit Cat No. BIO66NEPHA and Pi GST EIA Kit Cat No. BI085 which are available from Biotrin International Limited. α-GST levels≧11 μg/L indicate elevated αGST biomarker. Similarly, π-GST levels≧32 μg/L indicate elevated πGST biomarker.

Urine samples were collected from forty three ICU patients. Twenty one control urine samples were collected from healthy volunteers (Twelve males and nine females).

These samples were tested in a dot immunoblot assay, in triplicate, using antibodies PapX5C10 (IgG) for loop of Henle and HuPAPVII2B11 (IgM) for collecting duct as described in Preparatory Example H. Gold-labelled secondary conjugate antibodies were used to detect binding. The α and π-GST immunoassays were used to measure urinary α and 21-GST level in each sample.

### Results

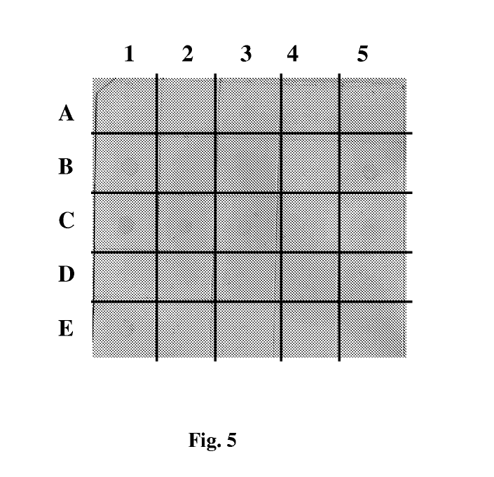

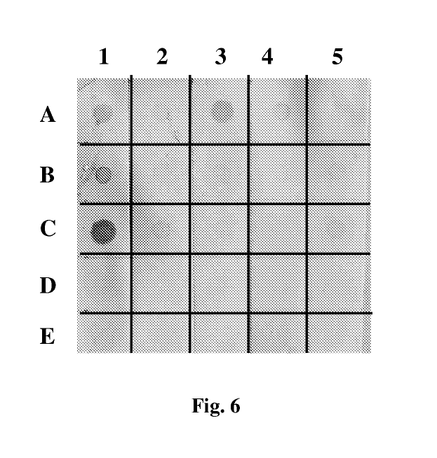

The results are set forth in Table 1 and are illustrated in FIGS. 5 and 6.

FIGS. 5 and 6 present immunoblot images using antibodies PapX5C10 or HuPAPVII2B11 as the detector molecule. It can be seen from these figures that antigen from the collecting duct is detectable in all of the kidney extract samples tested.

TABLE 1
Detection of biomarkers in urine obtained from ICU patients

| | | | PapX5 | Hu | pi GST | alpha GST |
|---|---|---|---|---|---|---|
| | | | C10 | PapVII2B11 | ug/L | ug/L |
| | | | loop of | Collecting | Distal | Proximal |
| Ref. | Sample Information | Origin | Henle | duct | Tubule | Tubule |
| A1 | Kidney Extract Lot 003 | Control 1/5 | + | +++ | | |
| A2 | Kidney Extract Lot 003 | Control 1/50 | − | + | | |
| A3 | Kidney Extract Lot 005 | Control 1/5 | + | +++ | | |
| A4 | Kidney Extract Lot 005 | Control 1/50 | − | ++ | | |
| A5 | PBS Buffer Control | — | − | − | | |
| B1 | Patient 6, Day 1 | ICU Patient | ++ | +++ | 254 | 24 |
| B2 | Patient 10, Day 1 | ICU Patient | − | + | 0 | 7 |
| B3 | Patient 10, Day 5 | ICU Patient | + | + | 23 | 26 |
| B4 | Patient 12, Day 5 | ICU Patient | − | + | 104 | 2 |
| B5 | Patient 14, Day 2 | ICU Patient | ++ | ++ | 14 | 8 |
| C1 | Patient 21, Day 1 | ICU Patient | +++ | +++++ | 28 | 1 |
| C2 | Patient 26, Day 4 | ICU Patient | ++ | ++ | 158 | 13 |
| C3 | Patient 27, Day 1 | ICU Patient | ++ | + | 28 | 19 |
| C4 | Patient 28, Day 1 | ICU Patient | − | + | 318 | 7 |
| C5 | Patient 34, Day 4 | ICU Patient | + | ++ | 174 | 9 |
| D1 | Male 1 | Normal | − | − | 9 | 9 |
| D2 | Male 2 | Normal | − | − | 26 | 15 |
| D3 | Male 7 | Normal | − | − | 9 | 4 |
| D4 | Male 9 | Normal | − | − | 12 | 7 |
| D5 | Male 11 | Normal | − | − | 14 | 6 |
| E1 | Female 1 | Normal | + | + | 37 | 5 |
| E2 | Female 4 | Normal | − | − | 12 | 3 |
| E3 | Female 6 | Normal | − | − | 6 | 2 |
| E4 | Female 8 | Normal | − | − | 4 | 3 |
| E5 | Female 9 | Normal | − | − | 21 | 8 |

Table 1 shows that as the concentration of kidney extract increased the binding signal increased as a result of the increase in the amount of collecting duct antigen available to the antibody.

Table 1 also shows that the PapX5C10 antibody was able to detect antigen from the loop of Henle at the highest concentrations of kidney extract (Samples A1 and A3).

Table 1 also indicates that no significant level of loop of Henle or collecting duct antigen present was present in any of the male volunteer urine samples. The πGST assay also showed no elevation in urinary πGST for any of the male volunteers. However, the αGST showed a slight elevation in male volunteer 2 (D2).

The buffer-only controls were negative in the collecting duct and loop of Henle assays.

The assay results indicate that one female volunteer (E1) had low levels of urinary loop of Henle antigen, collecting duct antigen and πGST levels. However, these low levels are not indicative of damage to these regions of the kidney tubule.

Patient 6, at Day 1, showed elevated levels of all four biomarkers indicating damage to the four main regions of the kidney tubule. The immunoblot signal for the urinary collecting duct antigen (B1) was similar to the kidney extract controls (A1 and A3). The loop of Henle signal (B1) was higher than that generated using the kidney extract controls (A1 and A3), suggesting significant kidney injury and a need for medical intervention.

Patient 10, at Day 1, showed the presence of a low concentration of collecting duct antigen which is not indicative of damage to this region. However, at Day 5, significantly elevated αGST biomarker was present in the patient's urine, indicating damage to the proximal tubule. The patient sample had low levels of loop of Henle and collecting duct antigen, but these levels are not indicative of damage to these regions.

Patient 12, at Day 5, showed significantly elevated urinary πGST levels. This indicates damage to the distal tubule. However, the injury had not extended to the loop of Henle, collecting duct or proximal tubule regions. Collecting duct antigen was present in the urine sample, but not at a significant concentration.

Patient 14, at Day 2, showed an elevated loop of Henle antigen and collecting duct antigen biomarkers. This indicates that damage was restricted to the loop of Henle and collecting duct regions.

Patient 21, at Day 1, had elevated loop of Henle antigen and collecting duct antigen biomarkers. This patient had the highest level of urinary loop of Henle antigen and collecting duct antigen producing the strongest PapX5C10 and HuPAPVII2B11 reactions. These reaction signals were considerably higher than that generated using the kidney extract controls as can be seen in FIG. 5 (C1) and FIG. 6 (C1). These high levels at this early stage (Day 1) indicate serious injury to these regions.

Patient 26, at Day 4, had significantly elevated levels of three biomarkers indicating damage to these regions of the kidney tubule. Urinary αGST biomarker was present but not at a concentration that would be indicative of damage to the proximal tubule.

Patient 27, at Day 1, showed elevated urinary loop of Henle antigen and αGST levels. This patient exhibited elevated biomarkers from two of the four main regions of the renal tubule at Day 1 in ICU, which indicates that this patient should be monitored for possible AKI development.

Patient 28, at Day 1, showed an elevated πGST biomarker level. The πGST level was the highest from the group tested (318 μg/L). This indicates major damage to the distal region. The fact that no other significant level of biomarker was observed indicates that the damage is restricted to this region.

Patient 34, Day, 4, shows an elevated level of collecting duct antigen and πGST biomarkers.

It is clear that if an individual αGST assay was performed on Patient 34, at Day 4, then the damage to the collecting duct and distal tubule would not have been detected. Results such as these indicate that the individual regions of the renal tubule can be damaged independently. This highlights the need for a panel, to determine the status of all the regions of the tubule.

Similarly, if α and πGST assays were the sole method of assessment of patients 14 and 21, the damage to the collecting duct and loop of Henle regions would be undetected and untreated. The use of the four biomarkers together presents an assessment of the four main regions of the kidney tubule.

### Example 3

### Preparation of Nitrocellulose Membrane with Capture Antibodies for Four Biomarkers Disposed Thereon

Predetermined regions of a nitrocellulose membrane are spotted with each of the four antibodies, 5B11 (binding to antigen in the proximal tubule), N5D12 (binding to antigen in the distal tubule), HuPapVII 2BII (binding to antigen in the collecting duct) and PapX5C10 (binding to antigen in the loop of Henle) in PBS containing 0.8% (w/v) BSA.

Excess binding sites on the membrane are blocked for 1 h with 1 ml blocking solution (PBS containing 4% (w/v) BSA). The wells are then washed in washing buffer (PBS) for 5 min.

Urine sample diluted ½ (1 ml) is added to the nitrocellulose membrane and incubated for 1 h. at room temperature followed by washing in wash buffer in triplicate.

Gold-labelled secondary conjugate antibodies for each of the biomarkers are diluted 1/100 in PBS containing 0.8% (w/v) BSA, 0.1% (w/v) gelatine hydrolysate and 0.05% (v/v) Tween® 20 in PBS and incubated with the nitrocellulose membrane for 2 h. at room temperature on a shaking platform.

After 2 hours incubation, the membrane is washed as follows
(i) 3×5 min washes with 0.8% (w/v) BSA in PBS on shaking platform.
(ii) 2×5 min washes with PBS on shaking platform.
(iii) 1×10 min incubation in 1% (v/v) glutaraldehyde in PBS (no shaking).
(iv) 2×5 min washes with dH₂O on shaking platform.
(v) 1×5 min wash with 50 mM EDTA pH 4.5 on shaking platform.

The nitrocellulose membrane is incubated for 30 min. at room temperature with BB International Silver Enhancing Kit (Cat. Code SEKL15) following the manufacturer's instructions. Development is performed without shaking.

The nitrocellulose membrane is extensively washed with distilled water.

1-24. (canceled)

25. A method of assessing kidney function in a human subject, which method comprises contacting a urine sample from said subject with a panel of at least three capture molecules, each capture molecule being capable of binding to a specific biomarker from at least one of the four major regions of the renal tubule, the detection of one or more biomarkers in the urine sample being indicative of damage in a corresponding region of the renal tubule.

26. A method according to claim 25, which is capable of detecting less than 60% damage in a region of the renal tubule.

27. A method according to claim 25, which is capable of detecting damage of the order of 5-10% in a region of the renal tubule.

28. A method according to claim 25, wherein the or each biomarker is detected by immunoassay.

29. A method according to claim 25, wherein the capture molecule is an antibody.

30. A method according to claim 29, wherein the antibody is a monoclonal antibody.

31. A method according to claim 29, wherein the antibody is monospecific for alpha glutathione S-transferase (αGST) specific to the proximal tubule.

32. A method according to claim 29, wherein the antibody is monospecific for pi glutathione S-transferase (πGST) specific to the distal tubule.

33. A method according to claim 29, wherein the antibody is monospecific for antigen specific to the collecting duct region of the renal tubule.

34. A method according to claim 29, wherein the antibody is monospecific for antigen specific to the loop of Henle region of the renal tubule.

35. A method according to claim 28, wherein the detection of the antibody biomarker complex comprises contacting the antibody-biomarker complex with a second antibody.

36. A method according to claim 35, wherein the second antibody is a labelled antibody and wherein the detection of the presence of biomarker-antibody complex is effected by detecting the label on the antibody.

37. A method according to claim 35, wherein the second antibody label is selected from the group consisting of an affinity label, biotin, a chromophore, a colloidal metal, dioxigenin, a dye, an enzyme, an enzyme substrate, a fluorophore, a lumiphore, a magnetic particle, a metabolite, a radioisotope and streptavidin.

38. A method according to claim 28, wherein the determination of the antibody-biomarker complex is carried out by a competition immunoassay.

39. A method according to claim 25, wherein a biomarker is detected enzymatically.

40. A method according to claim 25, wherein the capture molecules are bound to a microtitre plate.

41. A method according to claim 25, wherein the capture molecules are bound to a biochip array.

42. A method according to claim 25, for use in the diagnosis of diseases known to be associated with specific regions of the renal tubule.

43. A method according to claim 25, for use in assessing the effectiveness of therapeutic treatments.

44. A method according to claim 25, for use in assessing the tolerance of the subject to a specific pharmaceutical treatment.

45. A method according to claim 25, for use in assessing the safety of clinical trials.

46. A method according to claim 25, substantially as hereinbefore described and exemplified.

47. A test kit or assay comprising a panel of at least three_capture molecules, each capture molecule being capable of binding to a specific biomarker from at least one of the four major regions of the renal tubule.

48. A test kit or assay according to claim 46, substantially as hereinbefore described and exemplified.
